(19) United States
(12) Patent Application Publication
(10) Pub. No.: US 2010/0049137 A1
(43) Pub. Date: Feb. 25, 2010

(54) Device for Crossing Occlusions and Method of Use Thereof

(75) Inventor: Frank J. Fischer, JR., Bloomington, IN (US)

Correspondence Address: BRINKS HOFER GILSON & LIONE/CHICAGO/COOK, PO BOX 10395, CHICAGO, IL, 60610, US

(73) Assignee: Cook Incorporated, Bloomington, IN (US)

(21) Appl. No.: 12/194,584

(22) Filed: Aug. 20, 2008

(51) Int. Cl. A61M 25/09

(52) U.S. Cl. 604/164.13

(57) ABSTRACT

The present invention generally relates to a medical device and specifically to a catheter and wire guide for crossing an occlusion in a body lumen and a method of use thereof. In one embodiment, the catheter includes an incremental positioning mechanism positioned distally of the catheter body. In another embodiment, a lumen containing the wire guide extends through the incremental positioning mechanism and the catheter body. The incremental positioning mechanism allows for adjustment of the axial position of the wire guide relative to the distal end of the catheter body.

### TECHNICAL FIELD

The present invention generally relates to a medical device for crossing an occlusion within a body lumen and a method of use thereof.

### BACKGROUND

Effective treatments have been sought for disease conditions involving partial or total obstruction of a body vessel. Such conditions include, for example, narrowing or obstruction of the lumen of an artery (stenosis). This condition may be found in patients suffering from atherosclerosis, the accumulation of fibrous, fatty or calcified tissue in the arteries. An occlusion can result symptoms such as hypertension (high blood pressure), ischemia (deficiency of circulation), angina (chest pain), myocardial infarction (heart attack), stroke, or death. An occlusion may be partial or total, may be soft and pliable or hard and calcified, and may be found at a great variety of sites in the arterial system including the aorta, the coronary and carotid arteries, and peripheral arteries.

The treatment of occlusions occurring in the arteries supplying the heart is of particular interest to cardiac medicine. Traditionally, coronary artery occlusions have been treated by performing coronary bypass surgery, in which a segment of the patient's saphenous vein is taken from the leg and is grafted onto the affected artery at points upstream and downstream to the occlusion. Such a procedure often provides relief. However, it entails dangerous open chest surgery and a long convalescence. Moreover, with the passage of time, the patient's graft can also become occluded, requiring a second procedure.

Minimally invasive procedures are now preferred in the treatment of arterial occlusions. These procedures use a catheter, which is introduced into a major artery through a small arterial puncture made in the groin, upper arm, or neck and is advanced into the site of the occlusion. At the distal end of the catheter, a great variety of miniature devices have been developed for operating upon the occlusion.

Minimally invasive procedures include percutaneous transluminal coronary angioplasty (PTCA), directional coronary atherectomy (DCA), and stenting. PTCA employs a balloon to mechanically dilate the stenosis. In PTCA, a guidewire is introduced and advanced under fluoroscopic observation into the blocked artery and past the stenosis. A balloon-tipped catheter is then advanced over the guidewire until it is positioned across the stenosed segment. The balloon is then inflated, separating or fracturing the blockage.

Typically, the wire guide is positioned inside the inner lumen of an introducer catheter. The wire guide is advanced out of the distal end of the introducer catheter into the patient until the distal end of the wire guide reaches the location of the blockage. A major requirement for guidewires is that they have sufficient stiffness to be pushed through the patient's body lumen to the site of the occlusion, and then through the occlusion, without kinking. However, they must also be flexible enough to pass through the tortuous passageways without damaging the lumen wall. Efforts have been made to improve both the strength and the flexibility of wire guides to make them more suitable for their intended uses, but these two properties tend to be diametrically opposed to one another in that an increase in one usually involves a decrease in the other.

In some situations, a very flexible wire guide is required to pass through the tortuous passageways leading to the occlusion. Often, this wire guide is too flexible to push through the occlusion. This is particularly true then the occlusion is calcified. In these cases, the stiffer wire guide must be used. This requires that the wire guide is removed from the catheter and replaced by the stiffer wire guide. If the replacement wire guide is stiff not sufficiently stiff, this wire guide must be removed and progressively stiffer wire guides inserted until a wire guide sufficiently stiff to cross the occlusion is in place. This procedure may require the placement of five of more wire guides. In view of the above, it is apparent that there exists a need for an improved design for a device that does not require the use of multiple wire guides in such situations.

### BRIEF SUMMARY

One aspect provides a method of treating an occlusion within a body lumen. In one embodiment, the body lumen is a vascular vessel. A device including a catheter body and an incremental positioning mechanism positioned proximally of the catheter body is partially inserted into the lumen. A lumen containing a wire guide extends from the proximal end of the incremental positioning mechanism to the distal end of the catheter body. The incremental positioning mechanism allows for adjustment of the axial position of the distal end of the wire guide relative to the distal end of the catheter body.

The axial position of the wire guide relative to the distal end of the catheter body is adjusted to position the distal end of the wire guide beyond the distal end of the catheter body. The device is positioned so that the distal end of the wire guide is near the occlusion and is advanced distally to contact the distal end of the wire guide with the occlusion. The distal end of the wire guide is moved proximally with the incremental positioning mechanism to position the distal end of the wire guide closer to the distal end of the catheter body. The device is advanced distally, whereby the distal end of the wire guide crosses the occlusion.

Another aspect provides a device including a catheter body attached proximally to a first handle element having a threaded proximal portion. A second handle element having a threaded distal portion is positioned proximally of the first handle element. The threaded proximal portion of the first handle element engages the threaded distal portion of the second handle element. A lumen extends through the catheter body from a distal end of the catheter body and through the first handle element and the second handle element.

In one embodiment, the device also includes wire guide positioned within the lumen and extending from the distal end of the catheter body through the first handle element and the second handle element to the proximal end of the second handle element.

In another embodiment, the second handle element includes a locking mechanism allowing the axial position of a wire guide positioned within the lumen to be fixed. In yet another embodiment, the threaded proximal portion of the first handle element includes a threaded outside surface of the first handle element and the threaded distal portion of the second handle element includes a surface of the lumen extending through the second handle element. In yet another embodiment, the device includes graduated markings allowing observation of the relative axial positions of the first handle element and the second handle element.

### BRIEF DESCRIPTION OF THE DRAWINGS

FIG. 1 is an illustration of one embodiment of a combination catheter/wire guide.

FIG. 2 is a partial cross sectional view of a combination catheter/wire guide illustrating one embodiment of an incremental positioning mechanism.

FIG. 3 is a partial cross sectional view of a combination catheter/wire guide illustrating another embodiment of an incremental positioning mechanism.

### DETAILED DESCRIPTION

One aspect provides a method for crossing an occlusion or blockage in a body lumen of a patient. Another aspect provides a device including a catheter and a wire guide for use in medical procedures. As used herein, the term “proximal” refers to that portion of the device closest to a physician when placing the device in the patient, and the term “distal” refers to that portion of the device closest to the end inserted into the patient's body.

In one embodiment, the body lumen is a vascular vessel. For example, the method may be used to treat a narrowing or obstruction of the lumen of an artery (stenosis). Such obstructions may be found in patients suffering from atherosclerosis, the accumulation of fibrous, fatty or calcified tissue in their arteries. Such an occlusion may be partial or total, may be soft and pliable or hard and calcified, and may be found at a great variety of sites in the arterial system including the aorta, the coronary and carotid arteries, and peripheral arteries.

In one embodiment, a device including a catheter and a wire guide positioned within a lumen of the catheter is introduced into a major artery through a small arterial puncture made in, for example, the groin, upper arm, or neck and is advanced towards the site of the occlusion. The positioning of the device may be achieved with the assistance of one of more radiopaque marker(s) present within the device.

The catheter includes a catheter body and an incremental positioning mechanism at the proximal end of the catheter body. The incremental positioning mechanism allows for adjustment of the axial position of the distal end of the wire guide relative to the distal end of the catheter while the device is positioned in the body lumen. The stiffness of the distal and of the device may be decreased by moving the tip of the wire guide distally to increase the length of the wire guide extending beyond the distal end of the catheter. The stiffness may be decreased by moving the tip of the wire guide proximally to decrease the length of the wire guide extending beyond the distal end of the catheter. During placement of the device at the site of the occlusion, it is often preferable to minimize the stiffness or pushability of the distal end of the device to prevent damage to the lumen wall.

When positioned at the site of the occlusion, the catheter is advanced distally to contact the occlusion. If the occlusion is hard or calcified, the distal end of the wire guide may not be sufficiently stiff to penetrate and cross the occlusion. If this is so, the distal end of the wire guide is moved proximally by means of the incremental positioning mechanism to position the distal end of the wire guide closer to the distal end of the catheter body and by doing so to stiffen the distal end of the device. The device is then again advanced distally to contact the distal end of the wire guide with the occlusion. If the wire guide is still not sufficiently stiff to penetrate the occlusion, the length of wire guide extending beyond the distal end of the catheter body may again be reduced by adjusting the incremental positioning mechanism. Once the length of wire guide extending from the catheter body is sufficiently reduced to obtain sufficient stiffness, the device is advanced distally so that the distal end of the wire guide crosses the occlusion.

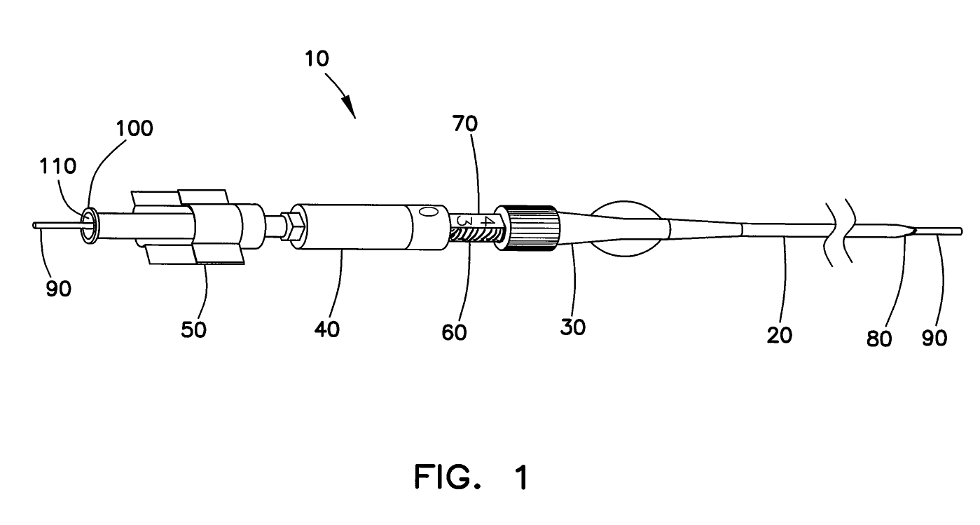

Referring now to FIG. 1, this figure illustrates one embodiment of a device for use in crossing an occlusion. The device includes a catheter and a wire guide positioned within a lumen of the catheter. Device 10 includes catheter body 20 and first handle element 30 attached proximally of catheter body 20. First handle element 30 includes a threaded proximal portion 60. Second handle element 40 is attached proximally to first handler element 30 so that a threaded distal portion of second handle element 40 engages threaded proximal portion 60 of first handle element 30. First handle element 30 and second handle element 40 form an incremental positioning mechanism allowing adjustment of the axial position of wire guide 90 relative to distal end 80 of the catheter body 20.

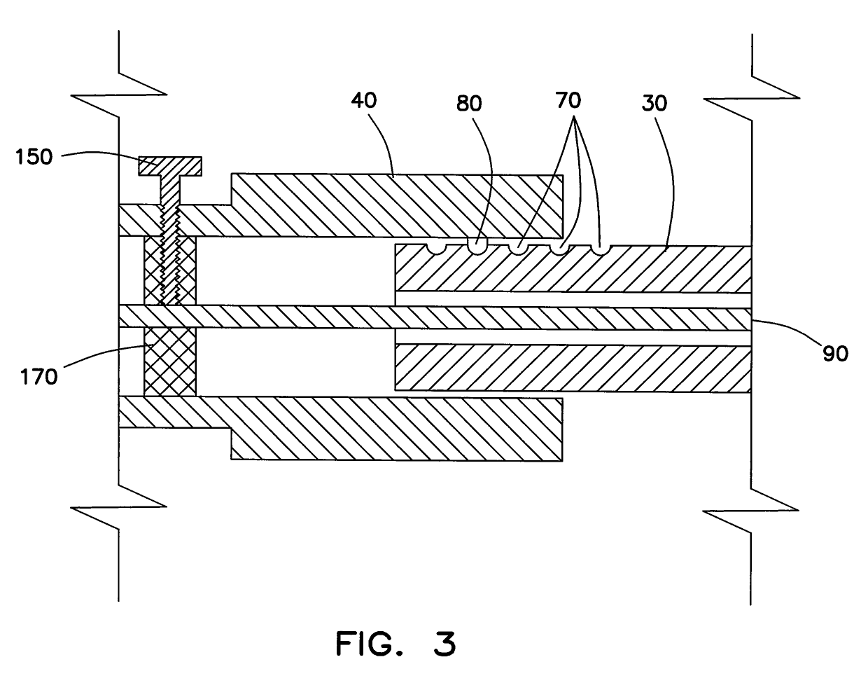

Lumen 110 extends from distal end 80 of catheter body 20 and through catheter body 20, first handle element 30 and second handle element 40 to proximal end 100 of second handle element 40. Wire guide 90 is positioned within lumen 110. In one embodiment, second handle element 40 includes locking mechanism 50 allowing the axial position of wire guide 90 to be fixed with respect to second handle element 40. For example, locking mechanism 50 can include a friction lock for engaging wire guide 90 in lumen 110 within second handle element 40. In one embodiment, the locking mechanism is a Touhy-Borst adapter. In another embodiment, the locking mechanism incorporates a collet. In yet another embodiment, such as that illustrated in FIG. 3, the locking mechanism includes screw 150 that engages wire guide 90 and holds it against extension 170.

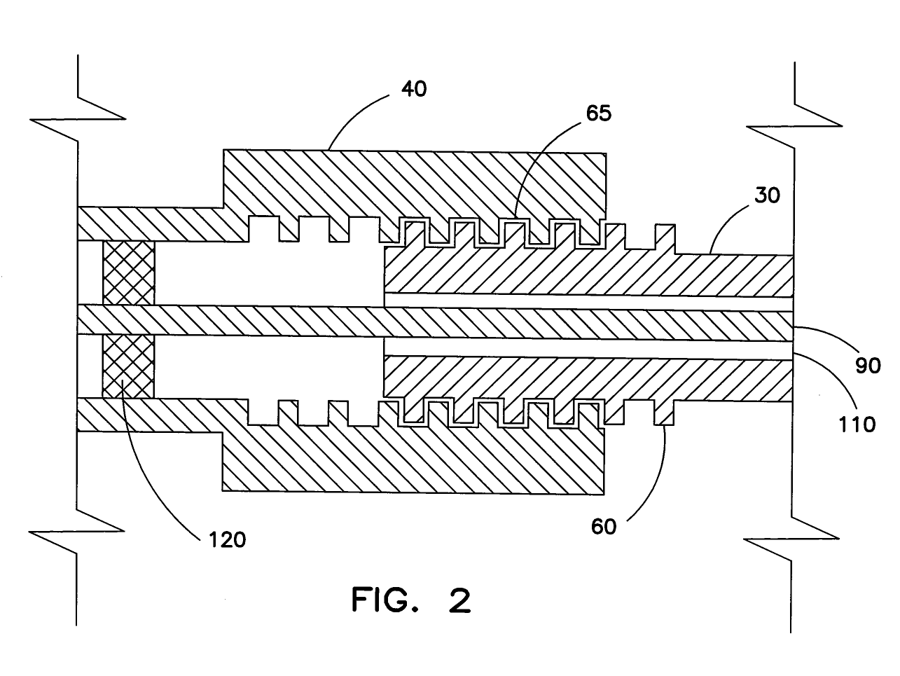

FIG. 2 illustrates one embodiment of the incremental positioning mechanism. In this embodiment, threaded proximal portion 60 of first handle element 30 is a threaded outside surface of first handle element 30 and threaded distal portion 65 of second handle element 40 is a surface of that portion of lumen 100 extending through second handle element 40. Of course, the present embodiments include an incremental positioning mechanism where the threaded proximal portion of the first handle element is a threaded portion of the lumen extending through the first handle element and the corresponding threaded distal portion of the second handle element is a threaded outside surface of the first handle element.

Wire guide 90 is positioned within lumen 110. The axial position of wire guide 90 may be fixed within second handle element 40 by engaging the locking mechanism to position element 120 against wire guide 90 so that the wire guide is not free to move axially with respect to second handle element 40. With the locking mechanism engaged, second handle element 40 may be rotated to move threaded distal portion 65, and wire guide 90, axially with respect to first handle element 30 and hence move the distal end of wire guide 90 axially with respect to the distal end of the catheter body.

FIG. 3 illustrates another embodiment of the incremental positioning mechanism. In this embodiment, the outside surface of first handle element 30 includes a number of indentations 70. The distal portion of second handle element 40 includes at least one protrusion 80 that engages successive indentations 70 as the first and second handle elements are moved axially relatively to each other. The spacing of the indentations 70 may be chosen to enable the operator to reproducibly move the two handle elements axially relative to each other by a fixed distance. The present embodiments also include an incremental positioning mechanism where the first handle element includes protrusions and the second handle element indentations.

In other embodiments, the incremental positioning mechanism includes both the screw mechanism illustrated in FIG. 2 and the protrusion/indentation mechanism illustrated in FIG. 3. In there embodiments, as the two handle elements are rotated with respect to each other they move axially relative to each other and successive protrusions or indentations are engaged.

In certain embodiments, the device also includes graduated markings indicating the relative axial positions of first handle element 30 and second handle element 40. Such markings allow for the accurate determination of the relative movement of the two handle elements and hence the relative change in position of the distal end of the wire guide with respect to the distal end of the catheter body. This allows for reproducible variation of the stiffness of the distal tip of the wire guide. For example, as is shown is FIG. 1, threaded proximal portion 60 of first handle element 30 may include graduated markings 70 allowing the relative axial positions of the first handle element and the second handle element to be observed.

Catheter body 20 may be formed from a material that supplies support for wire guide 90 and hence improves the pushability of the device through the body lumen. In one embodiment, catheter body 20 is formed from a polymer such as a polyurethane or a silicone. Catheter body may also include a marker at or near the distal end, thereby allowing the position of the distal end of the catheter to be determined while positioned within the vessel of a patient. For example, the marker may be a radiopaque marker. Suitable radio opaque markers are well known to those skilled in the art and include high-density metals such platinum or gold and materials such as barium sulfate. The wire guide may be radio opaque or may include a radio opaque marker positioned near the distal end to allow visualization of the position of the distal end of the wire guide.

In certain embodiments, the catheter body further includes a coating on at least a portion of the outer surface. The coating may include a material that reduces the coefficient of friction on that surface. For example, the coating may include a polymer such as, but not limited to, a fluoropolymer.

The wire guide may have typical wire guide dimensions. The wire guide length may generally be about 90 to about 300 cm, and for use within a patient's coronary system available wire guides are typically about 180 cm in length.

Although the invention has been described and illustrated with reference to specific illustrative embodiments thereof, it is not intended that the invention be limited to those illustrative embodiments. Those skilled in the art will recognize that variations and modifications can be made without departing from the true scope and spirit of the invention as defined by the claims that follow. It is therefore intended to include within the invention all such variations and modifications as fall within the scope of the appended claims and equivalents thereof.

We claim:

1. A method of treating a occlusion within a body lumen, comprising:
inserting a device into the body lumen, wherein the device comprises:
a catheter body and an incremental positioning mechanism positioned proximally of the catheter body, wherein a lumen extends from the proximal end of the incremental positioning mechanism to a distal end of the catheter body, and
a wire guide positioned within the lumen and extending from the proximal end of the catheter body to the distal end of the incremental positioning mechanism, wherein the incremental positioning mechanism allows for adjustment of the axial position of the wire guide relative to the distal end of the catheter body,
adjusting of the axial position of the wire guide relative to the distal end of the catheter body to position a distal end of the wire guide beyond the distal end of the catheter body,
positioning the distal end of the wire guide near the occlusion within the body vessel,
advancing the device distally to contact the distal end of the wire guide with the occlusion,
moving the distal end of the wire guide proximally with the incremental positioning mechanism to position the distal end of the wire guide closer to the distal end of the catheter body, and
advancing the device distally, whereby the distal end of the wire guide crosses the occlusion.

2. The method of claim 1, wherein the incremental positioning mechanism comprises:
a first handle element attached proximally of the catheter body and having a threaded proximal portion,
a second handle element positioned attached proximally to the first handle element and having a threaded distal portion, wherein the second handle element further comprises a locking mechanism allowing the axial position of the wire guide to be fixed within the lumen of the second handle element,
wherein the threaded proximal portion of the first handle element engages the threaded distal portion of the second handle element.

3. The method of claim 2, wherein moving the distal end of the wire guide proximally with the incremental positioning mechanism comprises:
fixing the axial position of the wire guide with respect to the second handle element by engaging the locking mechanism, and
rotating the second handle element with respect to the first handle element, whereby the second handle element moves in a proximal direction with respect to the first handle element.

4. The method of claim 1, further comprising observing the position of the distal end of the wire guide, wherein the wire guide further comprises a radio opaque marker positioned near the distal end to allow visualization of the position of the distal end.

5. The method of claim 1, wherein moving the distal end of the wire guide proximally with the incremental positioning mechanism to position the distal end of the wire guide closer to the distal end of the catheter results in an increase in pushability of that portion of the wire guide extending distally of the distal end of the catheter.

6. The method of claim 1, wherein moving the distal end of the wire guide proximally with the incremental positioning mechanism is performed at least two times during the advancement of the distal end of the wire guide across the occlusion.

7. The method of claim 2, wherein the threaded proximal portion of the first handle element comprises a threaded outside surface of the first handle element and the threaded distal portion of the second handle element comprises a surface of the lumen extending through the second handle element.

8. The method of claim 1, wherein the body lumen is a vascular vessel.

9. The method of claim 8, wherein the occlusion is calcified.

10. A device for treating an occlusion within a body vessel, the device comprising:
a catheter body,
a first handle element attached proximally of the catheter body, the first handle element having a threaded proximal portion, and
a second handle element attached proximally to the first handler element, the second handle element having a threaded distal portion, wherein the threaded proximal portion of the first handle element engages the threaded distal portion of the second handle element,
wherein a lumen extends through the catheter body from a distal end of the catheter body and through the first handle element and the second handle element.

11. The device of claim 10, the second handle element further comprising a locking mechanism allowing the axial position of a wire guide to be fixed within the lumen extending through the second handle element.

12. The device of claim 10, wherein the threaded proximal portion of the first handle element comprises a threaded outside surface of the first handle element and the threaded distal portion of the second handle element comprises a surface of the lumen extending through the second handle element.

13. The device of claim 10, further comprising graduated markings allowing observation of the relative axial positions of the first handle element and the second handle element.

14. A device for treating an occlusion within a body vessel, the device comprising:
a catheter body,
a first handle element attached proximally of the catheter body, the first handle element having a threaded proximal portion,
a second handle element attached proximally to the first handler element, the second handle element having a threaded distal portion, wherein the threaded proximal portion of the first handle element engages the threaded distal portion of the second handle element, and wherein a lumen extends through the catheter body from a distal end of the catheter body and through the first handle element and the second handle element, and
a wire guide extending within the lumen from the distal end of the catheter body through the first handle element and the second handle element to a proximal end of the second handle element.

15. The device of claim 14, further comprising graduated markings allowing observation of the relative axial positions of the first handle element and the second handle element.

16. The device of claim 14, the second handle element further comprising a locking mechanism allowing the axial position of the wire guide to be fixed within the lumen extending through the second handle element.

17. The device of claim 14, wherein the threaded proximal portion of the first handle element comprises a threaded outside surface of the first handle element and the threaded distal portion of the second handle element comprises a surface of the lumen extending through the second handle element.

18. The device of claim 17, wherein the threaded proximal portion of the first handle element further comprises graduated markings indicating the relative axial positions of the first handle element and the second handle element.

19. The device of claim 14, wherein rotation of the second handle element relative to the first handle element allows for adjustment of the axial position of a distal end of the wire guide relative to the distal end of the catheter body.

20. The device of claim 16, wherein the locking mechanism comprises a friction lock engaging the wire guide within the lumen of the second handle element.
